(19) United States
(12) Patent Application Publication
(10) Pub. No.: US 2021/0077285 A1
(43) Pub. Date: Mar. 18, 2021

(54) ABSORBABLE INTRAVASCULAR DEVICES FOR THE TREATMENT OF VENOUS OCCLUSIVE DISEASE

(71) Applicant: EFEMORAL MEDICAL INC., Los Altos, CA (US)

(72) Inventor: Lewis B. SCHWARTZ, Illinois (US)

(21) Appl. No.: 16/971,492

(22) PCT Filed: Feb. 22, 2019
(86) PCT No.: PCT/US2019/019300

§ 371 Date: Aug. 20, 2020

Related U.S. Application Data
(60) Provisional application No. 62/634,697, filed on Feb. 23, 2018.

(51) Int. Cl. A61F 2/90; A61F 2/958

(52) U.S. Cl. CPC A61F 2/90; A61F 2/958; A61F 2240/001; A61F 2250/0067; A61F 2230/0006; A61F 2210/0004

(57) ABSTRACT

A venous stent may be used to maintain or enhance patency of a blood vessel. By using multiple, separate stent elements that are balloon expandable, the multi-element stent may be stronger than a traditional self-expanding stent but may also be more flexible, due to its multiple-element configuration, than a traditional balloon-expandable stent. The stent elements are formed from a bioresorbable polymer material. The stent elements may have thick and/or wide struts and may be deployed oversized so as to overcome venous elastic recoil and anatomic compression.

### CROSS REFERENCES TO RELATED APPLICATION

This application claims the benefit and priority of U.S. Provisional Patent Application No. 62/634,697, entitled “ABSORBABLE INTRAVASCULAR DEVICES FOR THE TREATMENT OF VENOUS OCCLUSIVE DISEASE”, filed on Feb. 23, 2018, the full disclosure of the above referenced application is incorporated herein by reference.

### FIELD OF THE INVENTION

The present application pertains generally to the field of medical devices. More specifically, the present application pertains to the design and manufacture of intravascular stents intended to maintain patency (blood flow) of veins.

### BACKGROUND

Disturbance, compression, incompetence, stenosis and/or thrombosis of venous channels leads to a myriad of morbid human diseases affecting large proportions of the population. These diseases include chronic venous insufficiency of the lower extremities with varicose veins and skin ulceration (CVI), venous thromboembolism (VTE), venous outflow failure of hemodialysis access arteriovenous fistulas and grafts, Superior Vena Cava Syndrome (central venous occlusion), Paget-Schroetter Syndrome (upper extremity effort thrombosis), May-Thurner Syndrome (left iliac vein compression) and Nutcracker Syndrome (left renal vein compression).

Chronic venous insufficiency (CVI): Chronic venous insufficiency (CVI) is a term used to describe the phenomenon of deranged venous blood return from the lower extremities. Normal venous return is dependent on the patency and functionality of a series of large, compliant, interconnected veins that drain blood against the force of gravity through the use of a complex system of one-way valves driven by intermittent contraction of the skeletal muscles that surround them. Defects in the system, caused by primary incompetency of the valves, stenosis and/or clotting (deep venous thrombosis) increases ambulatory venous pressure leading to symptomology (pressure, fatigue, pain), leakage of proteinaceous serum into the subcutaneous tissue (edema), skin thickening and darkening (eczema and lipodermatosclerosis) and, in the end-stages, frank ulceration.

CVI is extremely common. Abnormal veins trouble >80% of the population; veins that are overtly dilated and varicose are found in 10-35%. Progression to venous hypertension and CVI is also exceedingly common and active or healed leg ulcers are present in 1-4% of the adult population. CVI is the single most common vascular disease.

The mainstay of treatment for CVI is compression hosiery which serves to mechanically limit venous dilation and control ambulatory venous hypertension and edema as it worsens throughout the day. Not surprisingly, patient compliance with these tight and unwieldy garments is poor. Recurrence is common, resulting in chronic disability and significant and consistent reductions in quality of life.

Superficial venous ablation (laser therapy) is a popular form of treatment for patients with saphenous and/or perforator valvular incompetence. It is particularly effective for patients with varicosity that is limited to the superficial system (unfortunately, a fairly uncommon disease pattern). The vast majority of patients with CVI exhibit involvement of the deep venous system, either with primary valvular incompetence of with post-phlebitic syndrome following DVT. Restoration of function of the deep venous system in patients with CVI has proven problematic. The few attempts at direct valve repair or replacement have not met with consistent success due to technical inadequacy and the ongoing thrombotic risk that follows operative manipulation or prosthetic device implantation.

Recently, it has been theorized that many patients with intractable CVI may have heretofore unrecognized chronic obstruction to venous outflow. The advent of advanced venous imaging has allowed for more precise, non-invasive morphologic assessment and, depending on the criteria utilized, some or many patients may be found to have either anatomical or physiological venous obstruction. Such occult venous stenoses can often be effectively treated with venous stenting, and large series with favorable outcomes have been reported. To date, the only devices available for this purpose are the large, metal stents that have developed for the endovascular treatment of arteries, the most popular being self-expanding stents made of either nitinol, stainless steel or elgiloy. Arterial stents used off-label in the venous system are problematic for several reasons including their generally inadequate mechanical dimensions (being designed and tested exclusively in the arterial system), their poor patency with high risks for restenosis and thrombosis, their unknown long-term fatigue resistance in the venous environment, their inadequate ability to withstand chronic mechanical compression, their risk of perforation and, most importantly, their disquieting tendency toward proximal migration into the right atrium.

Venous thromboembolism (VTE): Venous thromboembolism is the all-encompassing term applied to the clinical syndromes of deep venous thrombosis (DVT) and pulmonary embolism (PE). DVT occurs when one or more of the pathophysiologic triad of stasis of flow, blood hypercoagulability, and/or structural vascular defects causes clots to form in the deep veins of the pelvis and thigh. Some clots, for reasons that are unknown, break free from the deep veins, travel through the systemic venous system and lodge in the pulmonary arteries causing pulmonary embolism (PE), a condition with a high risk of immediate mortality.

Venous thrombosis is a ubiquitous problem, affecting 30-60 million inhabitants of developing countries annually. Among men, it is estimated that the cumulative probability of suffering a venous thromboembolic event (VTE) is 10.7% by the age of 80. Lower extremity deep venous thrombosis (DVT) complicates approximately 40-50% of strokes, elective hip replacements, multi-traumas, total knee replacements, and hip fractures, and 20-30% of myocardial infarctions, prostatectomies, spinal cord injuries, and neurosurgical operations. Pulmonary embolism (PE) arising from DVT is responsible for up to 200,000 deaths in the United States annually and remains the most common preventable cause of in-hospital mortality. PE causes more annual mortality than breast cancer, and has become the most frequent cause of death during childbirth.

Medical therapy with anticoagulation remains the mainstay of treatment for patients with VTE. Anticoagulation is highly effective in reducing the incidence of symptomatic PE, occurring in only 1-2% of adequately-dosed patients. It is generally held that anticoagulation prevents significant clot extension and/or symptomatic thrombosis of new venous segments, although some reports suggest that the incidence of ongoing subclinical thrombosis in patients receiving heparin may be as high as 20%. Also, medical therapy alone allows the obstructive clot to remain in situ indefinitely with its attendant long-term risks of CVI from post-phlebitic syndrome. CVI in some form will occur in 50-80% of patients following iliofemoral DVT, and symptoms will be severe in up to 30%. About 40% of all patients with venous ulcers have a history of thrombosis, and up to 70% have had ulcers in the past.

The most widely applied primary therapy for acute DVT is thrombolysis. Historically, chemical thrombolysis for DVT predated arterial applications. In early clinical trials, streptokinase was found to be superior to anticoagulation in promoting fibrinolysis, improving venous function, alleviating symptoms and reducing the incidence of PTS.

The need for efficient clot-directed delivery of thrombolytic agents and the rapid evolution of catheter-based technology led to the development of catheter-directed thrombolysis (CDT) as a strategy for DVT. CDT is performed by percutaneously gaining wire access to the thrombus and positioning a multi-holed catheter throughout its length to slowly infuse a thrombolytic agent. The results have largely been favorable, although the role of CDT in uncomplicated DVT remains a major debate within the field. Successful CDT dissolves clot, restores venous patency and reanimates valvular function. It works best when thrombolysis uncovers offending occlusive venous lesions which can then be treated with endovascular recanalization. Unfortunately, the only stents available for this application are large, metal stents that have developed for the endovascular treatment of arteries. The lack of an effective strategy for maintenance of venous patency in patients with long life expectancies represents a significant unmet clinical need.

Venous outflow failure of hemodialysis access fistulas and grafts: The filtering capacity of the kidneys is essential for human life; without it, death ensues in about a week. Kidney failure was uniformly fatal until the first hemodialysis machine, a filtering device that could temporarily assume the function of the kidneys by passing the patient's blood through a cellophane sack designed to draw out urea and other toxins.

Today, hemodialysis is ubiquitous. In the United States alone, over 700,000 patients are maintained on dialysis, with a total annual expenditure in excess of $30 billion (>5% of the entire CMS budget). Worldwide, it's estimated that more than 1.5 million people require dialysis to survive.

Approximately 70% of patients on hemodialysis maintain access to their vascular system via a peripheral arteriovenous fistula (AVF) or arteriovenous graft (AVG). These vascular reconstructive operations create high blood-flow conduits which can be repeatedly percutaneously accessed allowing patients' blood to rapidly flow though the dialysis machine to be filtered. Unfortunately, these vascular devices have proven notoriously difficult to maintain. Only about 60% of AVFs will mature to functionality71 and only about 50% of AVFs and 30% of AVGs will maintain primary patency for at least six months. Complications including venous outflow stenosis, frank thrombosis, pseudoaneurysm, infection and arterial steal are common; on average, patients will require at least one procedure every two years in order to keep blood reliably flowing through their access device.

The most common cause of failure of hemodialysis access fistulas and grafts is venous outflow stenosis. Outflow stenosis occurs as the fragile systemic vein is exposed to profound increases in blood flow, up to one-thousand fold, generating unpredictable and inconsistent velocity patterns which create boundary wall disturbance, shear stress aberration, inflammation, hypoxia, intimal hyperplasia and fibrosis. The mainstay of remediation therapy is percutaneous intervention, either with balloon angioplasty or metal stenting; unfortunately, neither is very well-suited to the pathobiology of the disease. Balloon angioplasty, while ubiquitous, rarely results in sustained patency, as the fibrotic vein recoils back its original diameter shortly following the procedure. In one study, the one-year primary patency of balloon angioplasty of venous outflow stenoses of dialysis access fistulas/grafts was a dismal 14%. In an attempt to overcome recoil, provide a wider flow channel and improve the results of angioplasty, metal stents are frequently implanted. However, intravascular metal stents designed for arterial indications are problematic in off-label, venous applications given their generally inadequate diameter, insufficient radial strength, propensity for intimal hyperplasia and restenosis, poor function in areas of upper extremity motion, and preclusion of percutaneous access through a metal device not intended for this purpose. Preservation of usable access is rarely sustained; clinical studies suggest that less than half of all percutaneous interventions will return functionality to the conduit for more than a year.

Theoretically, drug-eluting bioresorbable stents with high radial strength represent an attractive option for the treatment of failing dialysis access grafts. They could potentially provide firm scaffolding which could restore flow and resist venous recoil, attenuate restenosis allowing for prolonged patency and, most importantly, soften over time preserving venous movement and facilitating the repetitive percutaneous access required for ongoing dialysis.

Superior Vena Cava Syndrome (central venous occlusion): Obstruction of venous return through the superior vena cava and/or brachiocephalic veins is a serious and morbid clinical condition causing facial, neck and arm edema, dyspnea, cough and dilatation of subcutaneous veins. About 15,000 cases occur annually in the United States. The most common cause is malignancy, most notably bronchogenic carcinoma, but also small cell lung carcinoma, mesothelioma, lymphoma and leukemia. Benign SVC Syndrome, most frequently arising as a complication of chronic indwelling venous devices, is also fairly common. The syndrome is exceedingly morbid and difficult to treat; the average lifespan following the diagnosis is less than two years.

As anticoagulation rarely affords sufficient relief and surgical venous reconstruction is invasive and dangerous, endovascular intervention has emerged as the treatment-of-choice. Unfortunately, few intravascular devices are available to treat the large, central human veins, and none are approved for this purpose. Balloon angioplasty is the most commonly-employed technique, although venous rupture from overdilation has been reported and long-term results are spotty. Large, self-expanding stents are frequently implanted in an attempt to extend patency, but their short lengths, tendency toward foreshortening, generally inadequate diameters and poor resistance to external compression make achieving optimal results challenging.

The treatment of central venous occlusion and SVC Syndrome might be significantly enhanced with the availability of a balloon-expandable bioresorbable scaffold with high radial force. Balloon-expandable deployment of a sufficiently strong device would ensure accurate and permanent placement, and its absorbable nature would preserve the inherently thromboresistant properties of the venous endothelium.

Paget-Schroetter Syndrome (upper extremity effort thrombosis): Venous thoracic outlet syndrome progressing to the point of axilosubclavian vein thrombosis is variously referred to as Paget-Schroetter Syndrome, “effort thrombosis” or, sometimes, simply as “upper extremity deep venous thrombosis.” Its pathophysiology relates to compression of the axilosubclavian vein as it enters the thoracic at the costoclavicular junction. It is a relatively uncommon disorder; only about 3,000-6,000 cases occur in the United States annually. The condition is associated with significant morbidity, however, given its occurrence in young, active adults. Affected patients typically present with a blue, swollen, heavy extremity often with a history of vigorous exercise.

The optimal treatment of Paget-Schroetter Syndrome has been a matter of considerable controversy. In the current era, the treatment-of-choice consists of chemomechanical thrombolysis followed by anticoagulation and, after some interval of recovery, surgical thoracic outlet release via scalenectomy and first rib resection. The optimal treatment of the offending subclavian venous stenosis remains unknown. Although the vein could be safely and easily treated with stenting, many clinicians are reluctant to place a permanent metal device in an extrinsically-compressed vessel in a young patient. The availability of reliable, balloon-expandable absorbable stents would address this concern, allowing temporary support of the affected vein as the device is slowly resorbed.

May-Thurner Syndrome (left iliac vein compression): As stated above, deep venous thrombosis occurs when one or more of the pathophysiologic triad of stasis of flow, blood hypercoagulability, and/or structural vascular defects induces clots to form in large, deep veins. It has become increasingly recognized that many patients who present with left leg DVT have an identifiable anatomic variant that predisposes them to the syndrome. In normalcy, the left common iliac vein passes inferior to the right common iliac artery as it joins the confluence of the inferior vena cava. In some patients, the space between the right common iliac artery and the spine is unusually narrow, providing a “pinch point” of compression of the leftward-coursing left iliac vein. Although some measure of left iliac vein compression between the right common iliac artery and spine can be found in up to 25% of healthy people, those with minor venous abnormalities, bony protrusion, scoliosis and/or hypercoagulability are particularly prone to DVT. The prevalence has been difficult to estimate, although some have reported that May-Thurner Syndrome may be the cause of 18-49% of patients with left lower extremity DVT.

Patients presenting with left-sided DVT from May-Thurner Syndrome exhibit the same signs and symptoms of patients with acute DVT in general. However, many patients present prior to thrombosis; the chronic compression of the left iliac vein causing edema, pain in the pelvis or thigh, venous claudication and varicosity. Mechanical treatment of compression in these patients can afford significant symptomatic relief, as well as prevent the development of DVT. First-line treatment is endovascular with left iliac balloon angioplasty followed by self-expanding stenting. Complications are frequent, however, including venous rupture with retroperitoneal hemorrhage, the need for stenting the normal, contralateral right iliac vein or vena cava in order to hold the left iliac device in place, device migration, post-procedural pain from pressure on the spine and pelvis, late stent migration, recurrence of stenosis and/or thrombosis and post-phlebitic syndrome. Importantly, it's unknown whether intravascular devices intended for plaque-laden, non-compressed arteries can withstand decades of dynamic compression against the spine in patients that are relatively young.

Theoretically, the extrinsic venous compression of May-Thurner Syndrome could be effectively treated using a balloon-expandable, bioresorbable stent with high radial strength. The device would be secure in the space between the right common iliac artery and spine as it slowly softened and dissolved, remodeling the left common iliac vein to maintain patency.

Nutcracker Syndrome (left renal vein compression): Pelvic venous congestion syndrome (PVCS) is the combination of chronic pelvic pain and pelvic varicose veins. It has been variably known as PCVS, pelvic congestion syndrome, pelvic venous incompetence and pelvic varicose veins. Although its first clinical description appeared in the mid-nineteenth century, PVCS remains controversial and poorly understood to the present day. Opinions regarding PCVS vary widely, from skeptics and naysayers to devotees that believe the syndrome may affect 10% of all women of childbearing age and be responsible for a major segment of gynecological referrals for chronic pelvic pain.

It most commonly occurs in fertile females, although males can also be affected. The pain is generally dull, being variably described as throbbing, achy and/or heavy, and has usually been present for six months or more. It is typically located deep in the pelvis and groin, often with a slight predilection for the left side. Unlike the poorly localized pain generated by abdominal visceral afferent nerve fibers, patients with PVCS can often point to fairly specific regions of discomfort arising from venous dilatation. Associated pain in the vulva, upper thighs, left flank and/or lower abdominal quadrants is also common.

The symptoms of PCVS are generated by obstruction to pelvic venous flow. Three specific aberrant flow patterns have been identified: compression of the left renal vein between the superior mesenteric artery and aorta (Nutcracker Syndrome), compression of the left common iliac vein between the right common iliac artery and spine (May-Thurner Syndrome) and primary valvular reflux of the ovarian vein (primary gonadal venous reflux). May-Thurner is discussed above; primary reflux of the ovarian vein is not amenable to stenting so won't be discussed further.

As stated, Nutcracker Syndrome results from compression of the left renal vein between the superior mesenteric artery and aorta. As pressure in the left renal vein rises, the ostium of the left ovarian vein dilates and the fragile valve (if present) is rendered incompetent. Blood draining the kidney will reflux down the left ovarian vein into the pampiniform plexus. From there, it may travel into the perimetrial and myometrial veins of the uterus then return to the heart via the uterine or internal iliac veins. Flow across the midline is always pathologic. The diagnosis can be suggested by ultrasound, computed tomography and/or magnetic resonance venography, then confirmed by contrast venography which demonstrates compression of the left renal vein beneath the super mesenteric artery, an ovarian vein diameter >10 mm, uterine venous engorgement, congestion of the ovarian plexus, filling of pelvic veins across the midline, and/or filling of vulvovaginal thigh varicosities.

The treatment of choice for symptomatic Nutcracker Syndrome is endovascular recanalization of the compressed and obstructed left renal vein. Several series with favorable results have been reported. According to Chen et al., direct stenting of the left renal vein results in resolved or improved symptomology in >90% of affected patients.7 Care must be taken to size the device appropriately, however, as the increased blood flow post-procedure may stimulate venous dilatation resulting in stent dislodgement and migration. Furthermore, as the syndrome typically occurs in young females, the durability of metal stents in this dynamic anatomic location has been questioned. Implantation of a balloon-expandable, rigid, resorbable scaffold represents the ideal device design for this clinical application.

Limitations of intravascular metal stents in the venous system: As stated, several types of metal stents have been developed for implantation into human arteries. The first stent type to be widely applied was a balloon-expandable stent (BES) designed as an open mesh tube made of stainless steel. When crimped onto an angioplasty balloon it could be advanced through the arterial tree coaxially and deployed within the plaque by inflating the balloon. In the modern era, balloon-expandable stents are deployed in virtually every case of percutaneous coronary intervention (PCI) and in about half of all peripheral interventional procedures.

As early as 1969, it was theorized that intravascular stents should be more flexible than rigid. First developed for aerospace applications, an equiatomic alloy made of nickel-titanium called nitinol was thought to exhibit the ideal mechanical properties for the scaffolding of malleable blood vessels. One such property was superelasticity, or the ability of a metal to return to its original shape after a substantial deformation. This assured flexibility within arteries in motion within the human body. The other property was shape memory, or the ability of an alloy to be annealed at one temperature, substantially deformed at a lower temperature, then returned to its original shape when re-heated. This allows nitinol stents to be compressed into their delivery systems at low temperatures, then released and expanded within the warm mammalian environment at the time of implantation.

The first self-expanding nitinol stent to be approved for clinical use was a simple, coiled wire of nitinol. It was introduced into the American market in 1992. Seamless tubes of nitinol became available shortly after enabling the development of laser-cut, tubular nitinol stents.

Although neither balloon-expandable nor self-expanding stents were developed for venous applications, they are frequently utilized off-label as adjuncts to venous balloon angioplasty. Unfortunately, stents designed for arteries frequently perform poorly when implanted into veins. As opposed to arteries, veins increase in size in the direction of flow (“reverse taper”) such that vigorous venous flow tends to dislodge the stent with migration into the right atrium, right ventricle or pulmonary arteries. Veins exhibit profound diameter changes with respiration; stents are undersized will readily migrate. Venous stenting rarely results in sustained patency, as the fibrotic vein recoils back to its original diameter shortly following the procedure. Available stents diameters and lengths are rarely appropriate for veins, their radial strengths are insufficient, they have a propensity for intimal hyperplasia and restenosis, and they function poorly in areas of compression and/or bodily motion. Lastly, patients that require venous stents are often young in age; their indwelling metal devices will have to maintain their shape and function for decades. As metal stents designed for arteries are generally implanted in the elderly, their long-term fatigue properties are unknown and suspect.

Absorbable intravascular scaffolds: To address the myriad problems associated with permanent metal implants, stents that slowly dissolve after deployment have long been imagined. So-called “bioresorbable vascular scaffolds” (BVS) potentially offer several key biologic and physiologic advantages including, (1) effective scaffolding without the permanence of a metal implant, (2) attenuation of inflammation and chronic foreign body reaction leading to reduced restenosis and enhanced long-term patency, (3) assistance of adaptive vascular remodeling, (4) restoration of physiologic vasoactive function, and (5) facilitation of imaging and surveillance during follow-up.

The original bioresorbable device was the “catgut” surgical suture, first evident in the historical record some four millennia ago. Catgut sutures are derived from dried sheep, goat or bovine intestine, but have retained the name “catgut” probably because they were also used as strings for musical instruments sometimes referred to as “kits”. Catgut sutures are enzymatically degraded and resorbed in vivo so can be classified as bioresorbable. More contemporary bioresorbable surgical sutures are synthetic. Other, more recently developed bioresorbable medical devices includes bioresorbable screws and fracture plates for the treatment of traumatic injuries, indwelling scaffolds that serve as a basis for tissue engineering and regenerative medicine, chemotherapy-loaded polymers for therapeutic oncology, inert synthetic wraps for the prevention of post-operative peritoneal adhesions, bioabsorbable scaffolds for stenting of the upper airways and Eustachian tubes, and bioresorbable intravascular scaffolds (stents). Unfortunately, recent, more longer-term results have raised questions regarding the safety and efficacy of the first-generation absorbable coronary stent.

Therefore, it would be advantageous to have a stent for use in vasculature that is rigid upon implantation so as to maximally dilate and scaffold the vein, but then slowly decreases in rigidity to allow the blood vessel to return to its original, healthy, flexible state. At least some of these objectives will be met by the embodiments described below.

### SUMMARY

The embodiments herein describe a device for placement within a vein to maintain or enhance blood flow through the vein. The device may comprise multiple, balloon-expandable, bioresorbable, nenous stent elements configured to be implanted in the vein as a multi-element stent. The stent elements may be spaced such that the stent elements do not touch one another. The stent elements are formed from a bioresorbable polymer material. The stent elements may be configured to provide temporary, rigid, radial support to the vein following balloon angioplasty. The stent elements may have a thickness of approximately 250 microns or more. The stent elements may be formed by struts having a width of approximately 250 microns or more. In an embodiment, the stent elements comprise diamond shaped closed cells having circular keyhole shaped corners.

In some embodiments, the stent may be formed from a material comprising poly(L-lactic acid) (PLLA), poly(D-lactic acid) (PDLA), poly(D,L-lactic acid) (PDLLA), semicrystalline polylactide, polyglycolic acid (PGA), poly(lactic-co-glycolic acid) (PLGA), poly(iodinated desamino tyrosyl-tyrosine ethyl ester) carbonate, polycaprolactone (PCL), salicylate based polymer, polydioxanone (PDS), poly(hydroxybutyrate), poly(hydroxybutyrate-co-valerate), polyorthoester, polyanhydride, poly(glycolic acid-co-trimethylene carbonate), poly(iodinated desaminotyrosyl-tyrosine ethyl ester) carbonate, polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, or combinations thereof.

In an embodiment, the stent comprises a therapeutic drug. The therapeutic drug may prevent or attenuate inflammation, cell dysfunction, cell activation, cell proliferation, neointimal formation, thickening, late atherosclerotic change or thrombosis.

In an embodiment, the radial rigidity of the stent is slowly attenuated as its structural polymer is unlinked and metabolized such that the stent slowly becomes more flexible causing adaptation and remodeling of the vessel and restoration of the vessel's elasticity.

In an embodiment a method for maintaining or enhancing blood flow through a vein comprises implanting a balloon-expandable multi-element venous stent within a vein at a target location. The venous stent comprises multiple bioresorbable venous stent elements spaced such that the stent elements do not touch one another. The venous stent is expanded using a balloon to a diameter larger than the diameter of the vein at the target location. The stent elements may be formed from a bioresorbable polymer material. The stent elements may configured to provide temporary, rigid, radial support to the vein following implantation. The stent elements may have a thickness of approximately 250 microns or more. The stent elements may be formed by struts having a width of approximately 250 microns or more.

This and other aspects of the present disclosure are described herein.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1A illustrates one embodiment of a multi-element stent.

FIG. 1B is a magnified view of the stent elements in FIG. 1A.

FIG. 2A depicts deployment of a balloon-expandable multi-element stent.

FIG. 2B depicts deployment of a balloon-expandable multi-element stent.

FIG. 2C depicts deployment of a balloon-expandable multi-element stent.

FIG. 3A is a two-dimensional depiction of an element of a stent pattern.

FIG. 3B shows a magnified views of the cells in FIG. 3A.

FIG. 3C shows the stent element of FIG. 3A in cylindrical form.

FIG. 3D shows a magnified views of the cells in FIG. 3A.

FIG. 3E shows a magnified views of the cells in FIG. 3A.

FIG. 3F shows the stent element of FIG. 3A in cylindrical form.

FIG. 4A shows an embodiment of a stent pattern having diamond shaped cells with rounded corners.

FIG. 4B shows an embodiment of stent pattern having diamond shaped cells with circular keyhole shaped corners.

FIG. 4C shows an embodiment of stent pattern having diamond shaped cells with circular keyhole shaped corners.

FIG. 4D shows an embodiment of stent pattern having diamond shaped cells with circular keyhole shaped corners.

FIG. 5A is a two-dimensional depiction of an element of a stent pattern.

FIG. 5B shows a magnified view of the cells in FIG. 5A.

FIG. 5C shows the stent element of FIG. 5A in cylindrical form.

FIG. 5D shows the stent element of FIG. 5A in cylindrical form.

FIG. 6A show finite element analysis of a bioresorbable venous stent.

FIG. 6B show finite element analysis of a bioresorbable venous stent.

FIG. 6C show finite element analysis of a bioresorbable venous stent.

FIG. 6D show finite element analysis of a bioresorbable venous stent.

FIG. 6E show finite element analysis of a bioresorbable venous stent.

FIG. 6F show finite element analysis of a bioresorbable venous stent.

FIGS. 7A-7E show deployment of a segmented, rigid, absorbable scaffold in the right porcine iliofemoral vein.

FIG. 8 is a schematic diagram of a micro-stereolithograph used to create a stent, according to one embodiment.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Various embodiments are described herein with reference to the figures. The figures are not drawn to scale and are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

A typical “bioresorbable vascular scaffold” (BVS) or absorbable stent has a radial resistive force of under 2 N/cm. Similarly, a typical self-expanding metal stent (SES) has a radial resistive force of under 2 N/cm. Typical balloon-expandable metal stents (BES) have a much higher radial resistive force, sometimes above 18 N/cm.

The embodiments herein describe the design of a new, intravascular absorbable device that maintains the flow channel (patency) of blood vessels by providing temporary, rigid, radial support that is far greater than that provided by a typical absorbable or metal self-expanding stent (SES) and commensurate with that provided by a metal balloon-expandable stent (BES). Once implanted, the absorbable device imparts a high degree of radial force to prop open the diseased vein; the force is roughly equivalent to a large diameter, peripheral, balloon-expandable metal stent.

In contrast to most stent patterns which are designed to marry both radial force and longitudinal flexibility, the patterns described herein are specifically tailored to maximize radial force and rigidity and forego longitudinal and axial flexibility.

The devices described herein are multi-element, vascular stents (or “vascular scaffolds”). These stents are comprised of multiple, short, rigid, cylindrical stent segments, or elements, which are separate from one another but may be referred to together as a multi-element stent.

Generally, each element of the multi-element stents described herein will be sufficiently rigid to provide a desired level of strength to withstand the stresses of the vessel in which they are placed, such as a tortuous peripheral vessel. At the same time, a multi element stent will also be flexible, due to the fact that it is made up of multiple separate elements, thus allowing for placement within a curved, torturous blood vessel.

Additionally, the multi element stents described herein will usually be balloon-expandable rather than self-expanding, since balloon-expandable stents are typically stronger than self-expanding stents. Each balloon expandable element of the stent may have relatively high radial force (rigidity) due to the described structures and materials. A stent element is defined as being radially rigid if it has a radial strength significantly higher than self-expanding stents that is similar or greater in magnitude to that of traditional, metal balloon-expandable stents, such as those made of steel or cobalt-chromium.

When mounted serially on an inflatable balloon, they can be simultaneously implanted side-by-side in long blood vessels. During motion of the organism, the elements can move independently, maintaining their individual shape and strength while the intervening, non-stented elements of the vessel can twist, bend and rotate unencumbered. The result is a treated vessel with a rigidly maintained flow channel that still enjoys unrestricted flexibility during organismal movement.

The described embodiments exploit the principles that, (1) a rigid device that is deployed via balloon-expansion represents the optimal design of an intravascular stent given its transient effect on the venous wall and relative ease of precise implantation, (2) a long, rigid device cannot be safely implanted in an vein that bends and twists with skeletal motion, (3) long veins that bend and twist could be effectively treated with multiple, short BES that allow the intervening, non-stented venous elements to move unencumbered, (4) the length, number and spacing of the stent elements could be determined by the known and predictable bending characteristics of the target veins, and (5) veins need only be scaffolded transiently; late dissolution of the stent will have little effect on the long-term effectiveness of treatment.

Indicated for the treatment of long, occlusive lesions in bendable human veins, the device may be fashioned as a series of identical or near-identical rigid elements that are evenly spaced on a single, long balloon.

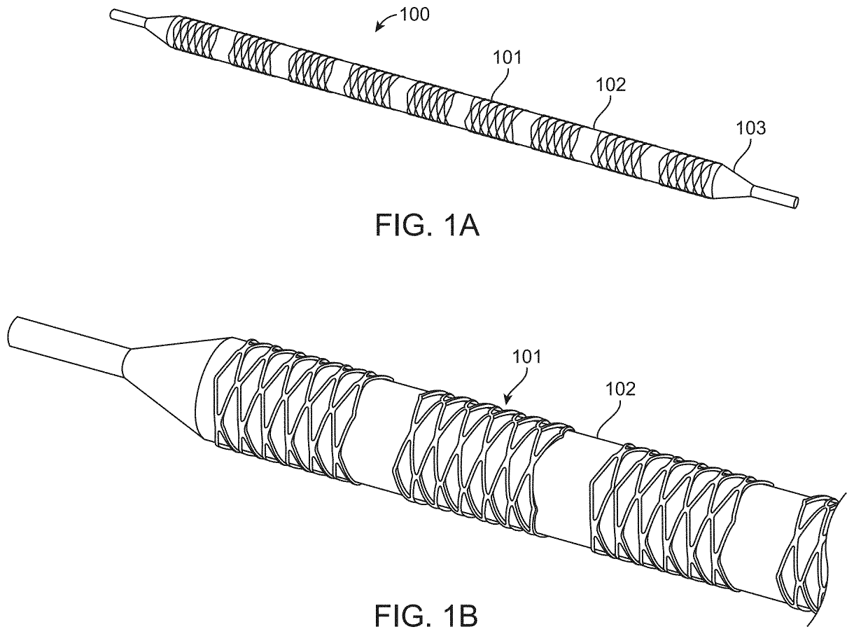

One embodiment of the fully assembled device in shown in FIG. 1A. A single balloon inflation and device deployment can treat a long segment of diseased vein while still preserving the critical ability of the vein to bend with skeletal motion such as sitting or walking. Multi-element stent 100 comprises multiple stent elements 101. Individual balloon-expandable stent elements 101 are crimped onto an inflatable balloon 103 to facilitate delivery. FIG. 1B is a magnified view of the stent elements 101 in FIG. 1A. Individual elements 101 are positioned serially along a longitudinal length of the balloon 103 and spaced such that the stent elements 101 do not touch one another. Further, the spacing is such that after deployment, the stent elements 101 do not touch or overlap during skeletal movement. The number of elements 101, length of elements 101, and gap 102 between elements 101 may vary depending on the target vessel location. In an embodiment, each element 101 in the multi-element stent 100 has the same length. In multi-element stents having three or more elements 101, and thus two or more gaps 102, the gaps may be of the same length.

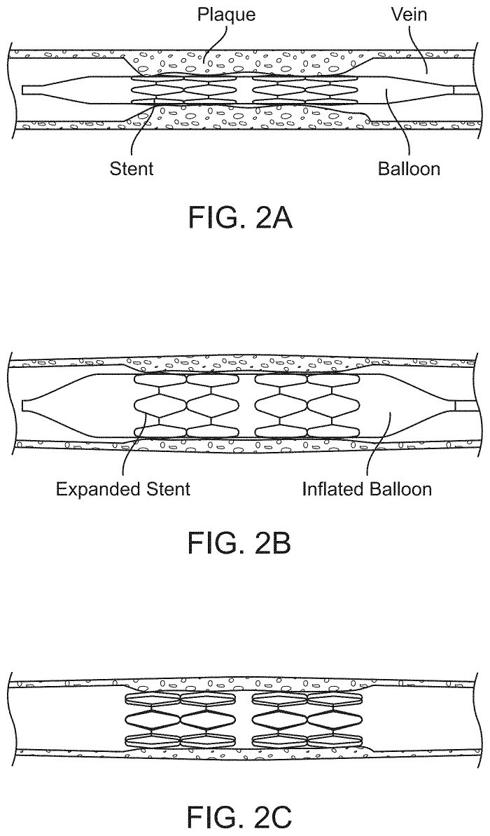

FIGS. 2A-2C depict deployment of a balloon-expandable multi-element stent. In FIG. 2A a multi-element stent mounted on a balloon is advanced to the lesion. In FIG. 2B the balloon and stent are expanded. In FIG. 2C the balloon is withdrawn leaving the multi-element stent still within the vein.

The stents described herein may be formed from various different materials. In an embodiment, stents may be formed a polymer. In various alternative embodiments, the stent or stent element may be made from any suitable bioresorbable material such that it will dissolve non-toxically in the human body, such as but not limited to poly(L-lactic acid) (PLLA), polyglycolic acid (PGA), poly(iodinated desaminotyrosyl-tyrosine ethyl ester) carbonate, or the like.

In alternative embodiments, any suitable polymer may be used to construct the stent. The term “polymer” is intended to include a product of a polymerization reaction inclusive of homopolymers, copolymers, terpolymers, etc., whether natural or synthetic, including random, alternating, block, graft, branched, cross-linked, blends, compositions of blends and variations thereof. The polymer may be in true solution, saturated, or suspended as particles or supersaturated in the beneficial agent. The polymer can be biocompatible, or biodegradable. For purpose of illustration and not limitation, the polymeric material may include, but is not limited to, poly(D-lactic acid) (PDLA), poly(D,L-lactic acid) (PDLLA), poly(iodinated desamino tyrosyl-tyrosine ethyl ester) carbonate, poly(lactic-co-glycolic acid) (PLGA), salicylate based polymer, semicrystalline polylactide, phosphorylcholine, polycaprolactone (PCL), poly-D,L-lactic acid, poly-L-lactic acid, poly(lactideco-glycolide), poly(hydroxybutyrate), poly(hydroxybutyrate-co-valerate), polydioxanone (PDS), polyorthoester, polyanhydride, poly(glycolic acid), poly(glycolic acid-co-trimethylene carbonate), polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, and combinations thereof. Non-limiting examples of other suitable polymers include thermoplastic elastomers in general, polyolefin elastomers, EPDM rubbers and polyamide elastomers, and biostable plastic material including acrylic polymers, and its derivatives, nylon, polyesters and expoxies. In some embodiments, the stent may include one or more coatings, with materials like poly(D,L-lactic acid) (PDLLA). These materials are merely examples, however, and should not be seen as limiting the scope of the invention.

Stent elements may comprise various shapes and configurations. Some or all of the stent elements may comprise closed-cell structures formed by intersecting struts. Closed-cell structures may comprise diamond, square, rectangular, parallelogrammatic, triangular, pentagonal, hexagonal, heptagonal, octagonal, clover, lobular, circular, elliptical, and/or ovoid geometries. Closed-cells may also comprise slotted shapes such as H-shaped slots, I-shaped slots, J-shaped slots, and the like. Additionally or alternatively, stent may comprise open cell structures such as spiral structures, serpentine structures, zigzags structures, etc. Strut intersections may form pointed, perpendicular, rounded, bullnosed, flat, beveled, and/or chamfered cell corners. In an embodiment, stent may comprise multiple different cells having different cell shapes, orientations, and/or sizes. In an embodiment, stent elements may comprise a plurality of diamond shaped closed cells longer in a longitudinal direction than in a radial direction when in an unexpanded state. The stent elements may also comprise a plurality of diamond shaped closed cells longer in a radial direction than in a longitudinal direction in the expanded state.

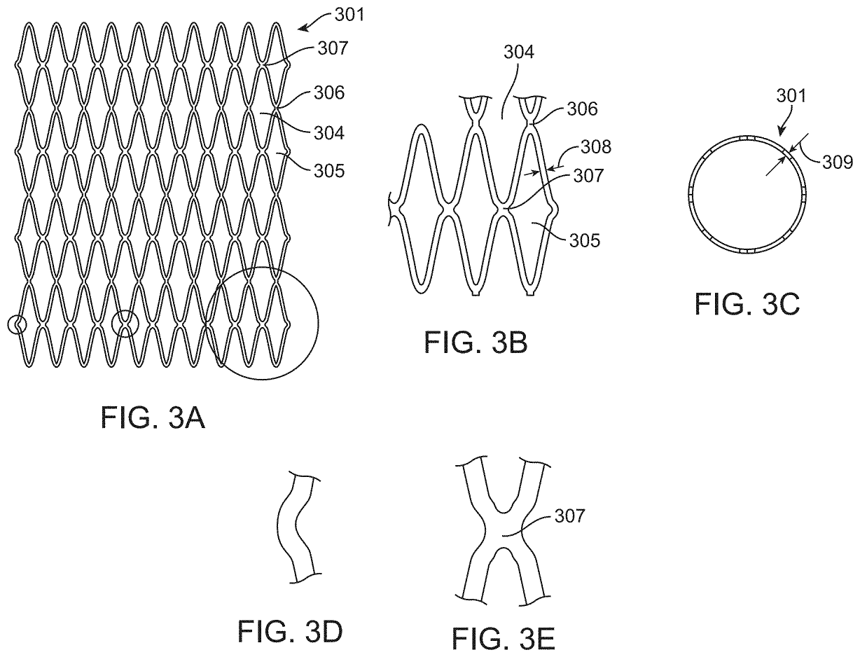

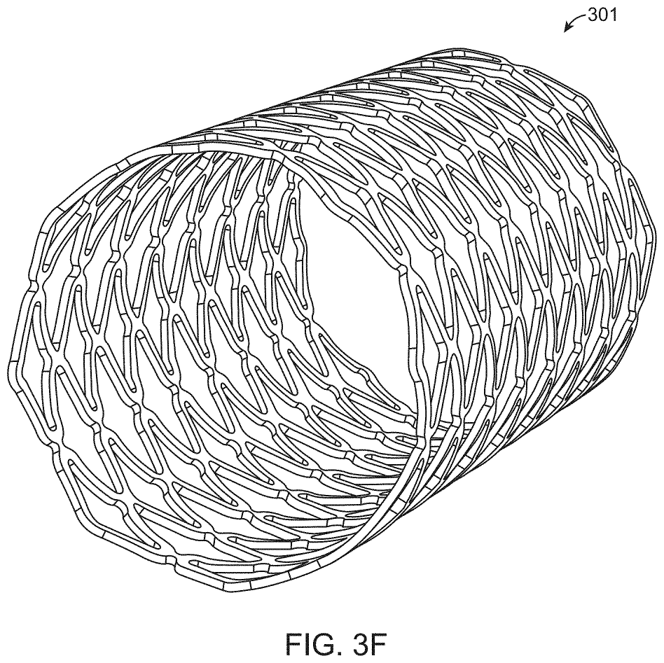

One embodiment of a stent pattern is shown in shown in FIGS. 3A-3F. The stent elements 301 have a diamond shaped closed-cell pattern. Elements 301 comprise intermixed diamond shaped closed cells 304, 305. Diamond shaped cells 304 may be aligned in the longitudinal and/or the circumferential directions in a repeating pattern. Similarly, diamond shaped cells 305 may be aligned in the longitudinal and/or the circumferential directions in a repeating pattern. Additionally or alternatively, diamond shaped cells 304 and diamond shaped cells 305 may be helically aligned in an alternating pattern. In an embodiment, diamond shaped cells 304 and diamond shaped cells 305 are circumferentially offset. Additionally, diamond shaped cells 305 may be formed at a central location between four adjacent diamond shaped cells 304. The width and/or the height of struts 306 between two corners of longitudinally aligned diamond shaped cells 304 may be larger or smaller than the width and/or height of struts 307 between two corners of longitudinally aligned diamond shaped cells 305.

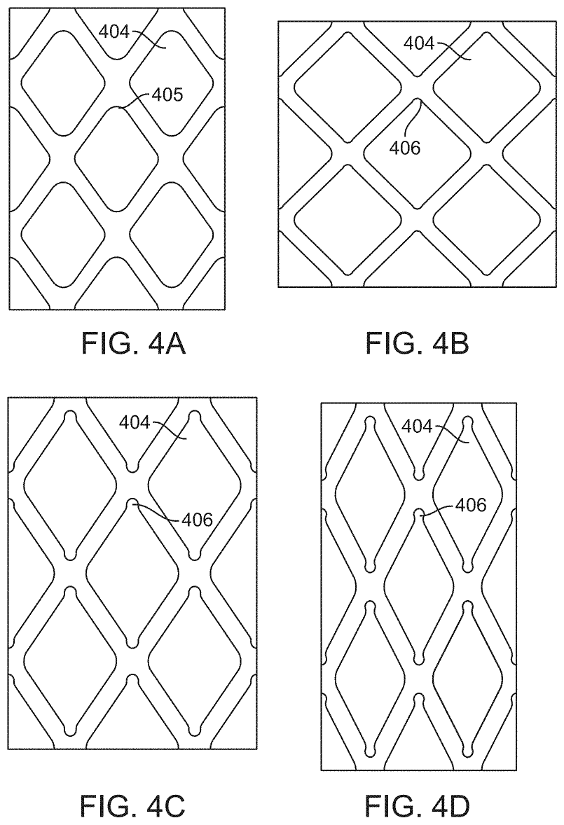

FIGS. 4A-4D show various embodiments stent patterns with diamond shaped closed-cell patterns. Diamond shaped cells 404 may have rounded corners 405. In various embodiments diamond shaped cells 404 may comprise circular keyhole shaped corners 406.

Unique characteristics of the scaffold patterns may include wide and/or thick struts and closed-cell structure designed for maximal strength without appreciable axial or bending flexibility (unlike metal stents). Thick and rigid scaffolds may be deployed oversized so as to overcome venous elastic recoil and anatomic compression, and remain securely imbedded within their venous target. Their high radial force, combined with the compressive force of the vein in which they are implanted, serves to specifically resist dislodgement and migration.

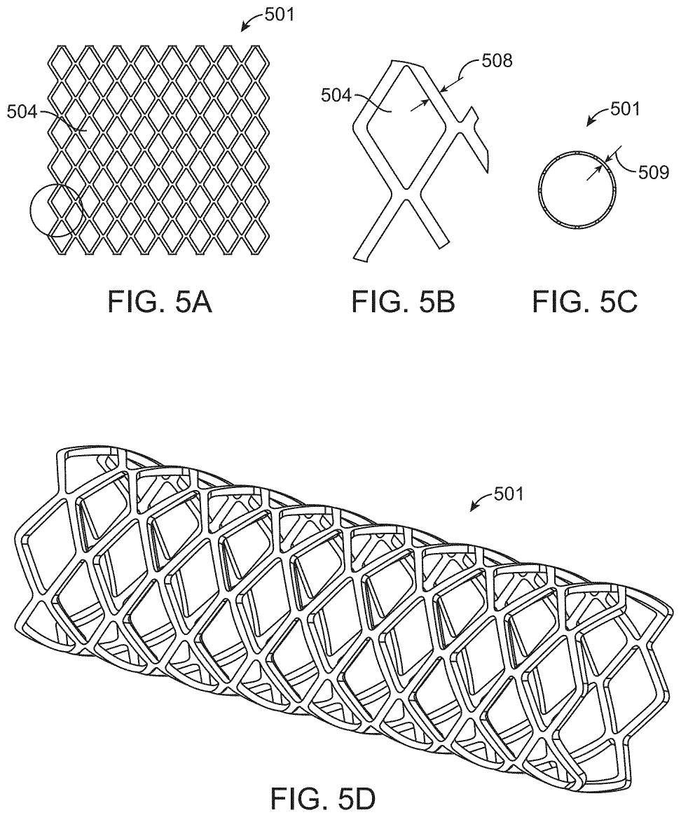

One embodiment of a stent pattern is shown in shown in FIGS. 5A-5D. The stent elements 501 have a diamond shaped closed-cell pattern. Elements 501 comprise diamond shaped closed cells 504. Elements 501 may comprise wide struts 506 of 225 microns or larger. Elements 501 may similarly comprise thick struts 506 of 225 microns or larger. In an embodiment, elements 501 comprise struts 506 with a width and/or thickness of approximately 250 microns. The width and/or the height of struts 506 between two corners of diamond shaped cells 304 may be larger or smaller than the width and/or height of struts 506 forming the sides of diamond shaped cells 304.

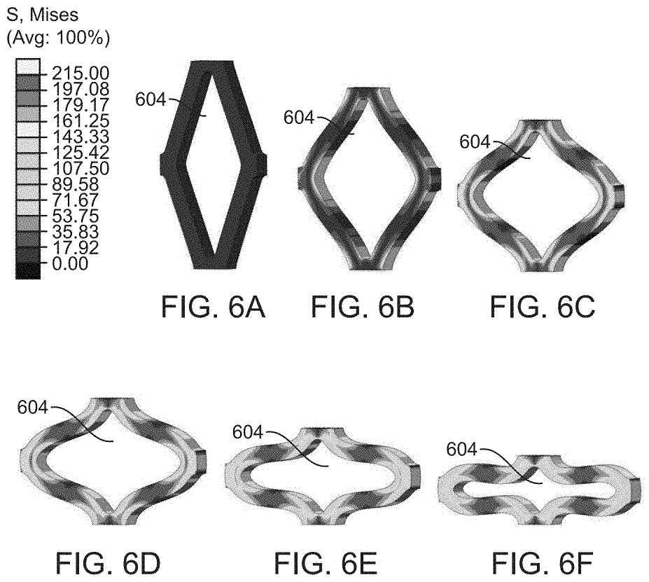

FIGS. 6A-6F show finite element analysis (FEA) of a bioresorbable venous stent showing thick struts than can withstand the stress of crimping. The stress scale is shown at the left. FIGS. 5A-5F show progressive crimping of a single cell 604. Note the maximal stress of 156 mises even when fully crimped (FIG. 6F) demonstrates that the device can be effectively crimped without undue strain or fracture.

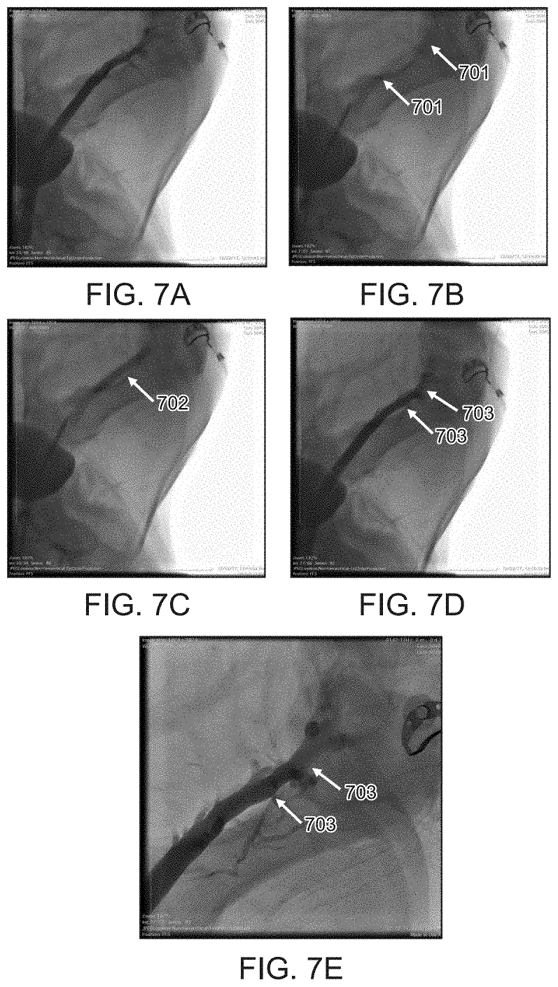

FIGS. 7A-7E show deployment of a segmented, rigid, absorbable scaffold in the right porcine iliofemoral vein. FIG. 7A shows a pre-procedure venogram via direct injection into the right iliofemoral vein. Device advancement is seen in FIG. 7B. The two segments of the device are located between the balloon markers 701. FIG. 7C shows device deployment via balloon inflation 702. FIG. 7D shows a fully deployed device; note the slight dilatation of the venous wall provided by the two scaffolds 703. FIG. 7E is a magnified view of the image in 7D.

The device described herein may include incorporation of a therapeutic drug intended to prevent or attenuate pathologic consequences of intraluminal intervention such as inflammation, cell dysfunction, cell activation, cell proliferation, neointimal formation, thickening, late atherosclerotic change and/or thrombosis. Any suitable therapeutic agent (or “drug”) may be incorporated into, coated on, or otherwise attached to the stent, in various embodiments. Examples of such therapeutic agents include, but are not limited to, antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cell adhesion promoters, antimitotics, antifibrins, antioxidants, anti-neoplastics, agents that promote endothelial cell recovery, matrix metalloproteinase inhibitors, anti-metabolites, antiallergic substances, viral vectors, nucleic acids, monoclonal antibodies, inhibitors of tyrosine kinase, antisense compounds, oligonucleotides, cell permeation enhancers, hypoglycemic agents, hypolipidemic agents, proteins, nucleic acids, agents useful for erythropoiesis stimulation, angiogenesis agents, anti-ulcer/anti-reflux agents, and anti-nauseants/anti-emetics, PPAR alpha agonists such as fenofibrate, PPAR-gamma agonists selected such as rosiglitazaone and pioglitazone, sodium heparin, LMW heparins, heparoids, hirudin, argatroban, forskolin, vapriprost, prostacyclin and prostacylin analogues, dextran, D-phe-pro-arg-chloromethylketone (synthetic anti-thrombin), glycoprotein IIb/IIIa (platelet membrane receptor antagonist antibody), recombinant hirudin, thrombin inhibitors, indomethacin, phenyl salicylate, beta-estradiol, vinblastine, ABT-627 (astrasentan), testosterone, progesterone, paclitaxel, methotrexate, fotemusine, RPR-101511A, cyclosporine A, vincristine, carvediol, vindesine, dipyridamole, methotrexate, folic acid, thrombospondin mimetics, estradiol, dexamethasone, metrizamide, iopamidol, iohexol, iopromide, iobitridol, iomeprol, iopentol, ioversol, ioxilan, iodixanol, and iotrolan, antisense compounds, inhibitors of smooth muscle cell proliferation, lipid-lowering agents, radiopaque agents, antineoplastics, HMG CoA reductase inhibitors such as lovastatin, atorvastatin, simvastatin, pravastatin, cerivastatin and fluvastatin, and combinations thereof.

Examples of antithrombotics, anticoagulants, antiplatelet agents, and thrombolytics include, but are not limited to, sodium heparin, unfractionated heparin, low molecular weight heparins, such as dalteparin, enoxaparin, nadroparin, reviparin, ardoparin and certaparin, heparinoids, hirudin, argatroban, forskolin, vapriprost, prostacyclin and prostacylin analogues, dextran, D-phe-pro-arg-chloromethylketone (synthetic antithrombin), dipyridamole, glycoprotein IIb/IIIa (platelet membrane receptor antagonist antibody), recombinant hirudin, and thrombin inhibitors such as bivalirudin, thrombin inhibitors, and thrombolytic agents, such as urokinase, recombinant urokinase, pro-urokinase, tissue plasminogen activator, ateplase and tenecteplase.

Examples of cytostatic or antiproliferative agents include, but are not limited to, rapamycin and its analogs, including everolimus, zotarolimus, tacrolimus, novolimus, and pimecrolimus, angiopeptin, angiotensin converting enzyme inhibitors, such as captopril, cilazapril or lisinopril, calcium channel blockers, such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, topoisomerase inhibitors, such as etoposide and topotecan, as well as antiestrogens such as tamoxifen.

Examples of anti-inflammatory agents include, but are not limited to, colchicine and glucocorticoids, such as betamethasone, cortisone, dexamethasone, budesonide, prednisolone, methylprednisolone and hydrocortisone. Non-steroidal anti-inflammatory agents include, but are not limited to, flurbiprofen, ibuprofen, ketoprofen, fenoprofen, naproxen, diclofenac, diflunisal, acetominophen, indomethacin, sulindac, etodolac, diclofenac, ketorolac, meclofenamic acid, piroxicam and phenylbutazone.

Examples of antineoplastic agents include, but are not limited to, alkylating agents including altretamine, bendamucine, carboplatin, carmustine, cisplatin, cyclophosphamide, fotemustine, ifosfamide, lomustine, nimustine, prednimustine, and treosulfin, antimitotics, including vincristine, vinblastine, paclitaxel, docetaxel, antimetabolites including methotrexate, mercaptopurine, pentostatin, trimetrexate, gemcitabine, azathioprine, and fluorouracil, antibiotics, such as doxorubicin hydrochloride and mitomycin, and agents that promote endothelial cell recovery such as estradiol.

Antiallergic agents include, but are not limited to, permirolast potassium nitroprusside, phosphodiesterase inhibitors, prostaglandin inhibitors, suramin, serotonin blockers, steroids, thioprotease inhibitors, triazolopyrimidine, and nitric oxide.

The beneficial agent may include a solvent. The solvent may be any single solvent or a combination of solvents. For purpose of illustration and not limitation, examples of suitable solvents include water, aliphatic hydrocarbons, aromatic hydrocarbons, alcohols, ketones, dimethyl sulfoxide, tetrahydrofuran, dihydrofuran, dimethylacetamide, acetates, and combinations thereof.

Stents may be manufactured using an additive or a subtractive. In any of the described embodiments, stents or stent elements may be manufactured as a sheet and wrapped into cylindrical form. Alternatively, stents or stent elements may be manufactured in cylindrical form using an additive manufacturing process. In an embodiment, stents maybe formed by extruding a material into a cylindrical tubing. In some embodiments, a longer stent element, may be formed during the manufacturing process and then cut into smaller stent elements/elements to provide a multi-element stent. In an embodiment, stent tubing may be laser cut with a pattern to form a stent element.

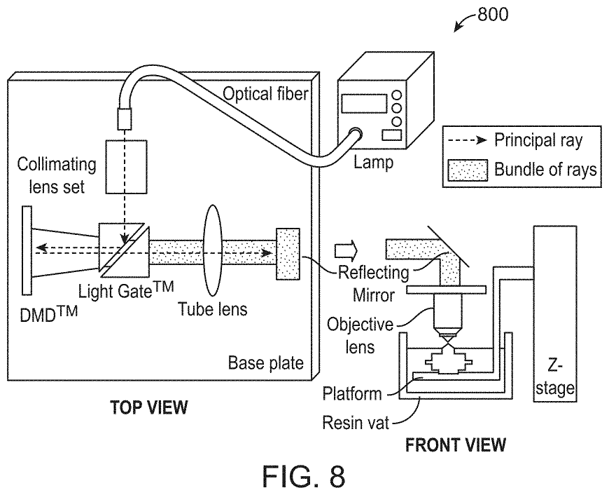

Referring now to FIG. 8, in one embodiment, stents may be manufactured using a micro-stereolithography system 800 (or “3D printing system”). Several examples of currently available systems that might be used in various embodiments include, but are not limited to: MakiBox A6, Makible Limited, Hong Kong; CubeX, 3D Systems, Inc., Circle Rock Hill, S.C.; and 3D-Bioplotter, (EnvisionTEC GmbH, Gladbeck, Germany).

The micro-stereolithography system may include an illuminator, a dynamic pattern generator, an image-former and a Z-stage. The illuminator may include a light source, a filter, an electric shutter, a collimating lens and a reflecting mirror that projects a uniformly intense light on a digital mirror device (DMD), which generates a dynamic mask. FIG. 8 shows some of these components of one embodiment of the micro-stereolithography system 800, including a DMD board, Z-stage, lamp, platform, resin vat and an objective lens. The details of 3D printing/micro-stereolithography systems and other additive manufacturing systems will not be described here, since they are well known in the art. However, according to various embodiments, any additive manufacturing system or process, whether currently known or hereafter developed, may potentially be used to fabricate stents within the scope of the present invention. In other words, the scope of the invention is not limited to any particular additive manufacturing system or process.

In one embodiment, the system 800 may be configured to fabricate stents using dynamic mask projection micro-stereolithography. In one embodiment, the fabrication method may include first producing 3D microstructural scaffolds by slicing a 3D model with a computer program and solidifying and stacking images layer by layer in the system. In one embodiment, the reflecting mirror of the system is used to project a uniformly intense light on the DMD, which generates a dynamic mask. The dynamic pattern generator creates an image of the sliced section of the fabrication model by producing a black-and-white region similar to the mask. Finally, to stack the images, a resolution Z-stage moves up and down to refresh the resin surface for the next curing. The Z-stage build subsystem, in one embodiment, has a resolution of about 100 nm and includes a platform for attaching a substrate, a vat for containing the polymer liquid solution, and a hot plate for controlling the temperature of the solution. The Z-stage makes a new solution surface with the desired layer thickness by moving downward deeply, moving upward to the predetermined position, and then waiting for a certain time for the solution to be evenly distributed.

Because the device is comprised of fully bioresorbable material, it slowly begins to weaken and dissolve soon after being subjected to a warm, biologically active environment. The device is designed such that its rigidity is slowly attenuated as its structural polymer is unlinked and metabolized. As the device weakens, its effect on the venous wall is slowly released. Eventually, the device ceases to exert any radial effect on its host vein thus completely removing any pathologic stimuli for neointimal hyperplasia formation, ongoing thickening and maladaptation. The lack of continuous stimulation by an intravascular foreign body allows the vessel to re-enter a quiescent, patent state until such time that further plaque might be generated by its host.

Although particular embodiments have been shown and described, they are not intended to limit the invention. Various changes and modifications may be made to any of the embodiments, without departing from the spirit and scope of the invention. The invention is intended to cover alternatives, modifications, and equivalents.

What is claimed is:

1. A device for placement within a vein to maintain or enhance blood flow through the vein comprising:
multiple, balloon-expandable, bioresorbable, venous stent elements configured to be implanted in the vein as a multi-element stent, wherein the stent elements are spaced such that the stent elements do not touch one another;
wherein the stent elements are formed from a bioresorbable polymer material;
wherein the stent elements are configured to provide temporary, rigid, radial support to the vein following balloon angioplasty;
wherein the stents elements have a thickness of approximately 250 microns or more; and
wherein the stent elements are formed by struts having a width of approximately 250 microns or more.

2. The device of claim 1, further comprising a therapeutic drug, wherein the therapeutic drug prevents or attenuates inflammation, cell dysfunction, cell activation, cell proliferation, neointimal formation, thickening, late atherosclerotic change or thrombosis.

3. The device of claim 1, wherein the bioresorbable polymer material comprises poly(L-lactic acid) (PLLA), poly(D-lactic acid) (PDLA), poly(D,L-lactic acid) (PDLLA), semi crystalline polylactide, polyglycolic acid (PGA), poly(lactic-co-glycolic acid) (PLGA), poly(iodinated desamino tyrosyl-tyrosine ethyl ester) carbonate, polycaprolactone (PCL), salicylate based polymer, polydioxanone (PDS), poly(hydroxybutyrate), poly(hydroxybutyrate-co-valerate), polyorthoester, polyanhydride, poly(glycolic acid-co-trimethylene carbonate), poly(iodinated desaminotyrosyl-tyrosine ethyl ester) carbonate, polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, or combinations thereof.

4. The device of claim 1, wherein the radial rigidity of the stent is slowly attenuated as its structural polymer is unlinked and metabolized such that the stent slowly becomes more flexible causing adaptation and remodeling of the vein and restoration of the vein's elasticity.

5. The device of claim 1, wherein the stent elements comprise diamond shaped closed cells having circular keyhole shaped corners.

6. A method for maintaining or enhancing blood flow through a vein comprising:
implanting a balloon-expandable multi-element venous stent within a vein at a target location, wherein the venous stent comprises multiple bioresorbable venous stent elements spaced such that the stent elements do not touch one another;
wherein the venous stent is expanded using a balloon to a diameter larger than the diameter of the vein at the target location;
wherein the stent elements are formed from a bioresorbable polymer material;
wherein the stent elements are configured to provide temporary, rigid, radial support to the vein following implantation;
wherein the stents elements have a thickness of approximately 250 microns or more; and
wherein the stent elements are formed by struts having a width of approximately 250 microns or more.

7. The method of claim 6, wherein the venous stent further comprising a therapeutic drug, wherein the therapeutic drug prevents or attenuates inflammation, cell dysfunction, cell activation, cell proliferation, neointimal formation, thickening, late atherosclerotic change or thrombosis.

8. The method of claim 6, wherein the bioresorbable polymer material comprises poly(L-lactic acid) (PLLA), poly(D-lactic acid) (PDLA), poly(D,L-lactic acid) (PDLLA), semicrystalline polylactide, polyglycolic acid (PGA), poly(lactic-co-glycolic acid) (PLGA), poly(iodinated desamino tyrosyl-tyrosine ethyl ester) carbonate, polycaprolactone (PCL), salicylate based polymer, polydioxanone (PDS), poly(hydroxybutyrate), poly(hydroxybutyrate-co-valerate), polyorthoester, polyanhydride, poly(glycolic acid-co-trimethylene carbonate), poly(iodinated desaminotyrosyl-tyrosine ethyl ester) carbonate, polyphosphoester, polyphosphoester urethane, poly(amino acids), cyanoacrylates, poly(trimethylene carbonate), poly(iminocarbonate), polyalkylene oxalates, polyphosphazenes, polyiminocarbonates, and aliphatic polycarbonates, fibrin, fibrinogen, cellulose, starch, collagen, polyurethane including polycarbonate urethanes, polyethylene, polyethylene terephthalate, ethylene vinyl acetate, ethylene vinyl alcohol, silicone including polysiloxanes and substituted polysiloxanes, polyethylene oxide, polybutylene terephthalate-co-PEG, PCL-co-PEG, PLA-co-PEG, PLLA-co-PCL, polyacrylates, polyvinyl pyrrolidone, polyacrylamide, or combinations thereof.

9. The method of claim 6, wherein the radial rigidity of the stent is slowly attenuated as its structural polymer is unlinked and metabolized such that the stent slowly becomes more flexible causing adaptation and remodeling of the vein and restoration of the vein's elasticity.

10. The method of claim 6, wherein the stent elements comprise diamond shaped closed cells having circular keyhole shaped corners.
